(19) United States
(12) Patent Application Publication
(10) Pub. No.: US 2018/0014741 A1
(43) Pub. Date: Jan. 18, 2018

(54) WEARABLE PHYSIOLOGICAL MONITORING DEVICE

(71) Applicant: Chang-An Chou, Taipei (TW)

(72) Inventor: Chang-An Chou, Taipei (TW)

(21) Appl. No.: 15/546,282

(22) PCT Filed: Jan. 25, 2016
(86) PCT No.: PCT/CN2016/072023

§ 371 Date: Jul. 25, 2017

(30) Foreign Application Priority Data
Jan. 26, 2015 (CN) 201510037884.2
Jan. 26, 2015 (CN) 201510037892.7
Jan. 26, 2015 (CN) 201510038012.8
Jan. 22, 2016 (CN) 201610043761.4
Jan. 22, 2016 (CN) 201620064146.7
Jan. 22, 2016 (CN) 201620064363.6
Jan. 22, 2016 (CN) 201620064809.5

(51) Int. Cl. A61B 5/0482; G09B 5/00; A61B 5/0205; A61B 5/0408; A61B 5/0478; G09B 19/00; A61B 5/00; A61B 5/024

(52) U.S. Cl. CPC A61B 5/0482; G09B 19/00; G09B 5/00; A61B 5/6803; A61B 5/0205; A61B 5/0408; A61B 5/0478; A61B 5/02416

(57) ABSTRACT

The present invention discloses a wearable physiological monitoring device for providing brain activity information and deciding a breathing guiding signal in a neurofeedback section, for being a basis for a user to perform a self-regulation about brain function. The device includes a wearable structure for mounting plural EEG electrodes and/or optical sensor on the head and/or an ear of the user so as to obtain information about brain activity and respiratory behavior.

### FIELD OF THE INVENTION

The present invention is related to a wearable physiological monitoring device, and particularly, related to a wearable physiological monitoring device used in a neurofeedback section.

### BACKGROUND OF THE INVENTION

Recently, more and more studies focus on how to influence the body functioning via self-regulation, for achieving an effect of improving physical and mental health. For example, biofeedback (including neurofeedback), meditation, and breath exercise etc. are all supported by many research results. There are more and more people using this kind of method.

Among theses, biofeedback is a process that enables an individual to learn how to change physiological activity for the purposes of improving health and performance. In this process, through the individual's will, e.g., thought, emotion and behavior, there may have physiological changes, e.g., brainwaves, heart rate, breathing, muscle activity, and skin temperature, which could be measured by instruments, and then, the information is feedback to the individual rapidly and accurately. Because this information is in conjunction with the target physiological changes, the individual can perform the self-regulation based on the received information, so as to enhance the desired physiological changes.

Neurofeedback is a type of biofeedback that provides user real-time information about brain activity. Electroencephalography (EEG) is most commonly used to represent brain activity. After receiving the information about brain activity in real time, through self-regulation, the user will be able to achieve the influence on brain function.

Moreover, another important application of EEG is to be used as brain computer interface (BCI), wherein through detecting EEG, it will be able to analyze the intentions of user, and the intentions will be converted into operation commands. Recently, this kind of BCI cooperating with neurofeedback is also applied to games, for example, a game for training concentration.

Consequently, when it is related to employ the regulation mechanism of human body to improve physical and mental health, or to BCI applications, the self-regulation is the most important means. As known, concentrating is one of the main approaches to perform self-regulation. Therefore, if the concentration can be improved during the neurofeedback process so as to help the performance of self-regulation, the target of neurofeedback will be achieved more efficiently.

Generally, during the meditation which also needs to concentrate, it usually will emphasis that the mediator must focus on the rhythm of breathing, especially when the mediator's mind becomes wandering, the concentration must be re-focused on the rhythm of inspiration and expiration. Thus, focusing on breathing rhythm is the known way to improve concentration.

Generally, without consciousness, the breathing is controlled by autonomic nervous system (ANS) which will automatically regulate the respiration rate and depth in accordance with body's demands. On the other hand, breathing also can be controlled by consciousness. Within a limited rage, human can control the respiration rate and depth. Therefore, as shown in the researches, through controlling the respiration, it will be able to influence the balance of sympathetic nervous system (SNS) and parasympathetic nervous system (PSNS). In general, during expiration, the PSNS activity is increased and the heart rate is slowed down; and during inspiration, the SNS activity is increased and the heart rate is risen up.

Thus, when focusing on the breathing rhythm, except of achieving concentration and stabilization, it will also have influence on ANS. At this time, if the influence of breathing on ANS is identical to the target of neurofeedback, such as, relaxation, the effect of neurofeedback will be leveled up due to the increment of breathing control. The two processes are mutually complementary.

Consequently, there indeed has a need to develop a novel system which, during performing the neurofeedback via self-regulation, can provide the user a basis for adjusting the respiration thereof, so that the influence of breathing on improving physical and mental health can also be revealed simultaneously. Consequently, through the mutual complementary between the two processes, the overall effect can be leveled up.

### SUMMARY OF THE INVENTION

One object of the present invention is to provide a wearable physiological monitoring device, for providing brain activity information and deciding a breathing guiding signal in a nurofeedback section, for being a basis for a user to perform a self-regulation about brain function, thereby achieving a neurofeedback loop. The device includes plural electroencephalographic (EEG) electrodes; a heart rate sensing unit; a wearable structure, combined with the plural EEG electrodes and the heart rate sensing unit, wherein when the wearable structure is mounted on the head and/or an ear of the user, the plural EEG electrodes are located on the head and/or the ear where are capable of achieving an EEG signal acquisition loop, and the heart rate sensing unit is located on the head and/or the ear where is capable of acquiring heart beat sequence; and a physiological signal acquisition circuitry, for acquiring EEG signals via the plural EEG electrodes, and obtaining the heart beat sequence via the heart rate sensing unit. In the neurofeedback section, the EEG signals are used to be a basis for generating brain activity related information for providing to the user; the heart beat sequence is used to perform a respiratory behavior related analysis so as to obtain an analysis result, and the analysis result is then used as a basis to provide and/or adjust the breathing guiding signal; and the user performs a self-regulation based on the brain activity related information and performs a respiratory behavior based on the breathing guiding signal, so that commonly through the self-regulation about brain and through the respiratory behavior influences autonomous nervous system (ANS), an influence on brain function is achieved.

Another object of the present invention is to provide wearable physiological monitoring device including plural EEG electrodes, which are implemented to be dry electrodes, an optical sensor, an ear-worn structure including an ear-plug structure or an ear-hook structure, and an ear-clamp structure, wherein the ear-plug structure or the ear-hook structure is configured to mount on an ear of a user, and the ear-clamp structure is configured to clamp an ear portion of the ear, a physiological signal acquisition circuitry, for acquiring EEG signals via the plural EEG electrodes, and obtaining heart rate via the optical sensor, wherein at least one of the plural EEG electrodes and the optical sensor are mounted on the inner side the ear-clamp structure together, and at least another of the plural EEG electrodes is mounted on the ear-plug structure or the ear-hook structure, and when the ear-clamp structure is configured to clamp on the ear portion, the EEG electrode at the inner side contacts the skin of the ear portion to achieve an EEG signal acquisition loop, and the optical sensor acquires heart beat sequence from the ear portion.

Another object of the present invention is to provide a wearable physiological monitoring device including plural EEG electrodes, an optical sensor, an ear-plug structure and an ear-hook structure, wherein the ear-plug structure is configured to mount inside an ear of a user and the ear-hook structure is configured to hook on the ear, and a physiological signal acquisition circuitry, for acquiring EEG signals via the plural EEG electrodes, and obtaining heart rate via the optical sensor, wherein at least one of the plural EEG electrodes is mounted on the ear-plug structure, and at least another of the plural EEG electrodes is mounted on the ear-hook structure, the optical sensor is mounted on the ear-plug structure, and when the ear-plug structure and the ear-hook structure are mounted on the ear simultaneously, the plural EEG electrodes contacts the skin of the ear and/or around the ear for achieving an EEG signal acquisition loop, and the optical sensor acquires heart beat sequence from the ear.

Another object of the present invention is to provide a wearable physiological detection device including plural EEG electrodes, an optical sensor, an ear-plug structure, mounted inside an ear of a user, and a physiological signal acquisition circuitry, for acquiring EEG signals via the plural EEG electrodes, and obtaining heart rate via the optical sensor, wherein at least two of the plural EEG electrodes and the optical sensor are mounted on the ear-plug structure, and when the ear-plug structure is mounted inside the ear, at least one of EEG electrodes which are mounted on the ear-plug structure is configured to contact the canal of the ear, the opening of canal, and/or the inferior concha of the ear for achieving an EEG signal acquisition loop, and the optical sensor acquires heart beat sequence from the ear.

A further object of the present invention is to provide a wearable physiological detection device which is capable of acquiring EEG signals and heart beat sequence for being used in a neurofeedback section.

A further object of the present invention is to provide a wearable physiological detection device which, in a neurofeedback section, is capable of providing brain activity information for a user to perform self-regulation and of deciding a breathing guiding signal based on the user's respiratory behavior for being followed by the user, so as to achieving an influence on brain function.

Another further object of the present invention is to provide a wearable physiological detection device which is mounted on a user's head via a head-worn structure, such that EEG electrodes are arranged at positions capable of achieving an EEG signal acquisition loop and the optical sensor is located at a position capable of acquiring heart beat sequence.

Another further object of the present invention is to provide a wearable physiological detection device which is mounted on a user's ear via an ear-worn structure, such that EEG electrodes are arranged at positions capable of achieving an EEG signal acquisition loop and the optical sensor is located at a position capable of acquiring heart beat sequence.

Still another object of the present invention is to provide a wearable physiological detection device which obtains a user's heart rate and respiratory behavior through analyzing the user's heart beat sequence, so as to, in the neurofeedback section, provide information about the correlation among EEG signals, respiratory behavior and heart rate, to the user for being the basis to perform self-regulation.

Still another object of the present invention is to provide a wearable physiological detection device which obtains a user's brain activity information and also respiratory behavior both from EEG signals, so as to, in the neurofeedback section, provide the brain activity information for the user to perform self-regulation, and take the respiratory behavior as the basis to provide and/or adjust a breathing guiding signal.

Still another further object of the present invention is to provide a wearable physiological detection device, in which plural EEG electrodes and the optical sensor are both mounted on an ear-worn structure, so as to acquire EEG signals and heart beat sequence simultaneously when the device is worn on the ear.

Still another further object of the present invention is to provide a wearable physiological detection device, in which the optical sensor and one of the plural EEG electrodes are positioned in an ear clamp structure for clamping on a portion of an ear.

### BRIEF DESCRIPTION OF THE DRAWINGS

FIG. 1 shows the operation of a wearable physiological detection device which is mounted on a head of a user via a head-worn structure in a preferred embodiment according to the present invention;

FIG. 2 shows the wearable physiological detection device of FIG. 1 with an additional ear-worn structure;

FIGS. 3A-3C show examples of ear clamp structure;

FIGS. 4A-4D shows wearable physiological detection devices whose ECG electrode is mounted at different portions of user's body;

FIGS. 5A-5B show shows wearable physiological detection devices whose ECG electrode is located at an exposed surface thereof;

FIGS. 6A-6B show wearable physiological detection devices which are mounted on the head via a glasses structure of preferred embodiments according to the present invention;

FIGS. 7A-7B show wearable physiological detection devices which are mounted on an ear of the user via an ear-worn structure of preferred embodiments according to the present invention;

FIGS. 8A-8C show examples of wearable physiological detection devices which employ ear-worn structure for being mounted on the ear and also have ECG electrode to acquire ECG signals;

FIG. 9 shows an example of wearable physiological detection device which employs ear-worn structure for being mounted on the ear and also includes EEG electrodes, ECG electrodes and optical sensor;

FIG. 10 shows the physiological structure of an auricle; and

FIG. 11 is a schematic view showing the positions of the cortex and the auricle.

### DETAILED DESCRIPTION OF THE INVENTION

The object of the present invention is to integrate a process of self-regulation for influencing brain activity and a process of breathing regulation into one single neurofeedback section, and through the interaction with the user to form a neurofeedback loop, so as to enhance the influence on brain activity, thereby leveling up the effect that can be achieved by the processes.

Under this principle, the wearable physiological monitoring device according to the preset invention is configured to equip with at least two electroencephalographic (EEG) electrodes and a heart rate sensing unit, wherein the EEG electrodes are used to acquire EEG signals for revealing the brain activity, and the heart rate sensing unit is used to acquire heart beat sequence for being the basis of providing and/or adjusting the breathing guiding signal.

Generally, for acquiring EEG signals, it will need at least two electrodes, wherein one is used as active electrode and the other is used as reference electrode. Further, it is also familiar to add one more ground electrode for suppressing common mode noises, such as, 60Hz and 50Hz noises. Therefore, in the following, the descriptions will mainly focus on two EEG electrodes only, the active electrode and the reference electrode.

Further, as known, breathing will influence autonomic nervous system (ANS) and thus cause variations in heart rate, which is controlled by ANS. This is the so called Respiratory Sinus Arrhythmia (RSA), which means the heart beat will increase during inspiration and will decrease during expiration. Therefore, it will be able to reveal the respiratory behavior through detecting the heart rate. Generally, when the breathing and the heart beat are in a synchronization state, it will be able to obtain the respiratory behavior through analyzing the heart beat sequence. In the present invention, the sensing unit for obtaining the heart beat sequence can be implemented as optical sensor or ECG electrodes. The optical sensor means the sensor having light emitting element and light receiving element and acquiring optical signals based on PPG (photoplethysmography) principle. The optical sensor can obtain the heart beat sequence through detecting the continuous pulse variations, such as, via transmission or reflectance manner. The ECG electrodes can be used to acquire electrocardiogram, and thus, obtain the heart beat sequence.

Further, the obtained the heart beat sequence also can be used to perform HRV (Heart Rate Variability) analysis. HRV analysis is a common way to observe ANS activity. For example, it can perform a frequency domain analysis, so as to obtain TP (Total Power) for evaluating overall HRV, HF (High Frequency Power) for representing PSNS activity, LF (Low Frequency Power) for representing SNS activity or the result of simultaneous adjustments of PSNS and SNS, and LF/HF for representing the activity balance between SNS and PSNS. Further, after frequency domain analysis, through observing the frequency distribution, the harmony of SNS also can be revealed. Alternatively, it also can perform a time domain analysis, so as to obtain SDNN for being the indication of overall HRV, SDANN for being the indication of long term overall HRV, RMSSD for being the indication of short term overall HRV, and R-MSSD, NN50 and PNN50 for evaluating the middle to high frequency variation. Accordingly, it will be able to know the influence of neurofeedback and/or respiratory regulation on the ANS through analyzing the heart beat sequence.

Therefore, in the concept of the present invention, brain activity related information, ANS activity related information and respiratory behavior are mutually complementary and can be used to provide user a more comprehensive and effective neurofeedback process, so as to maximize the effect that can be achieved by self-regulation. Further, if the optical sensor is used, it will be able to additionally obtain the information about oxygen saturation, which is helpful for further understanding the user's physical condition.

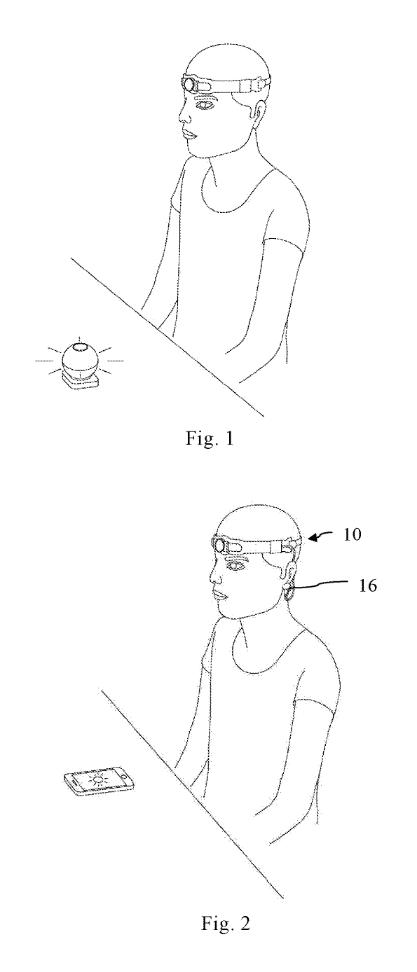

In practice, as shown in FIG. 1, the wearable physiological monitoring device 10 of the present invention is mounted on a user's head via a head-worn structure 14 with EEG electrodes and optical sensor. The device 10 includes a main unit 12 having a physiological signal acquisition circuitry contained therein for acquiring physiological signals through EEG electrodes and optical sensor. Thus, the physiological signal acquisition circuitry will include, but not limited, the common electronic elements for measurement, such as, processor, at least an A/D converter, filter, amplifier etc., which are common for one skilled in the art and thus not described here.

Moreover, two EEG electrodes are positioned onto the user's head through the head-worn structure. The EEG electrodes can be mounted at the inner side of the head-worn structure for contacting the sampling points on the head for acquiring EEG signals. For example, the commonly used sampling points include Fp1, Fp2, O1, O2, or any position in accordance with the international 10-20 system. Here, the position and number of EEG electrodes should be decided by the purpose of neurofeedback and not limited, for example, the number of active electrode can be increased to perform multi-channel EEG measurement.

In the present invention, preferably, the EEG electrodes are implemented to be dry electrodes, which could be made from all kind of metals or conductive materials, such as, stainless steel, conductive fiber, conductive rubber, conductive foam, and conductive gel etc. Accordingly, the EEG signals can be acquired through a direct contact of electrode with the skin of head, so that the problems derived from using the traditional wet electrode, such as, the necessity of conductive gel and adhesive tape, could be avoided. Therefore, not only the operation becomes more convenient, but the user also will be more willing to use it. The head-worn structure also can be implemented to be different types. For example, the head-band as shown in FIG. 1, or other types, such as, a headgear which is commonly used in a general EEG measurement, or a pair of glasses. It only needs to be a structure that can be positioned on the head and can ensure the contact between the electrode and the skin, for example, the common used head-worn structure usually has a shape circling the skullcap so as to facilitate the electrodes to be located at sampling points corresponding to cortexes. Thus, there is no limitation.

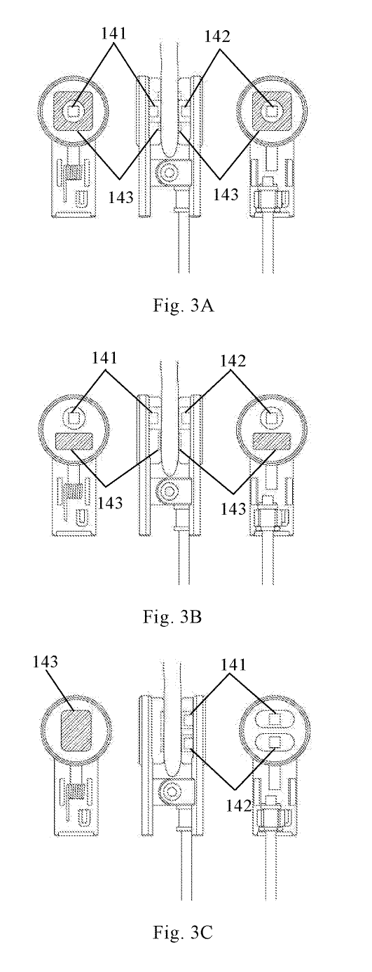

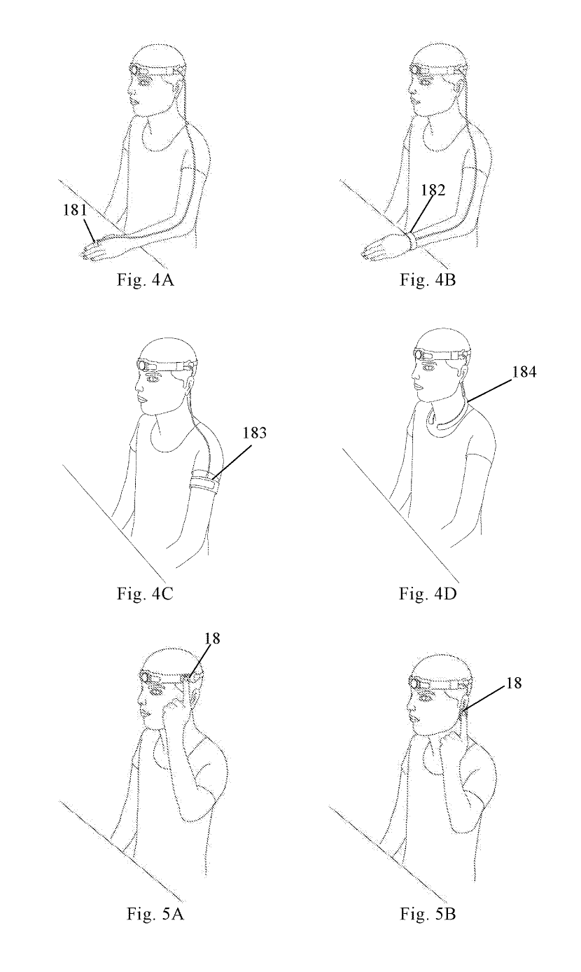

The optical sensor, through the head-worn structure, also can be positioned at any location of the head, such as, to contact the forehead for acquiring continuous pulse variations. Alternatively, as shown in FIG. 2, the optical sensor also can be extended from the head-worn structure via a connecting wire so as to be mounted on an ear for acquiring the continuous pulse variations conveniently. And, based on the position and operation, it can be selected to detect pulses in a transmission or a reflectance manner.

When the optical sensor is configured to mount on the ear, it also can be implemented to be carried by an ear-worn structure, such as, ear-clamp structure (as the ear clamp 16 in FIG. 2), ear-hook structure or ear-plug structure, so as to locate on the ear or around the ear, such as, ear lobe, the concave side of auricle, e.g., inferior concha, the area near the opening of canal, and/or helix, the convex side of auricle, the canal, and/or the area near the junction between the ear and the scalp, without limitation. By employing suitable ear-worn structure, the optical sensor can be fixed better, thereby improving the stability and also signal quality.

Preferably, one of the EEG electrodes also can be configured to locate on the ear-worn structure. In the field of EEG measurement, ears are usually regarded as suitable positions to place reference electrode due to the discontinuous physiological structure and position from the head, which makes ears not easy to be influenced by brain activity. Thus, to combine the reference electrode with the ear-worn structure for contact the skin of the ear or the area near the ear not only is beneficial of acquiring EEG signal with good quality, but also can keep the whole configuration simple, which is advantageous.

For example, FIG. 3A shows an ear clamp which can be installed conveniently and can achieve a stable contact. As shown, the optical sensor is implemented to be a light emitting element 141 and a light receiving element 142 respectively mounted at the opposite surfaces inside the ear clamp. An EEG electrode 143 is also mounted in the ear clamp at where capable of contacting the skin of the ear. Accordingly, through the mechanical force of the clamp, no matter the optical sensor or the EEG electrode, both can be set on the ear stably and not easily be moved, thereby facilitating to acquire signals of good quality and also to obtain accurate analysis results.

When the optical sensor and EEG electrode are mounted in the ear clamp at the same time, the arrangement thereof can have different choices. For example, as shown in FIG. 3A, the EEG electrode can be implemented to surround the light emitting element/light receiving element, or as shown in FIG. 3B, the EEG electrode also can be implemented to separate from the light emitting element/light receiving element. Further, it also can be implemented to set electrode at both side of the ear clamp for being reference electrode and ground electrode. Alternatively, the EEG electrode also can be set at one side only for being the reference electrode. There is no limitation. Alternatively, as shown in FIG. 3C, the light emitting element 141 and the light receiving element 142 can be set at the same side of clamp, and in this case, the reflectance manner will be used to measure heart rate. As to the EEG electrode, it can be set at the other side of clamp.

It should be noted that the ear clamp can be implemented to clamp any portion of the ear, namely, any portion of the auricle, such as, the ear lobe, or the helix, without limitation. And, the mechanical structure thereof also can be changed in accordance with the ear portion clamped.

Accordingly, while the user performs the neurofeedback, the physiological monitoring device of the present invention can be located on the head via the head-worn structure (and on the ear via the ear-worn structure), and at the same time, the installation of electrodes and optical sensor can be completed simply. Then, the EEG signals acquired via EEG electrodes will be processed based on a preset algorithm to obtain the brain activity related information, which is provided to the user for being a basis to perform self-regulation. And, the heart beat sequence acquired via the optical sensor also will be processed based on the algorithm to obtain the respiratory behavior related information for being a basis to provide and/or adjust the breathing guiding signal.

Then, please refer to FIG. 4A, which shows the wearable physiological monitoring device of the present invention which employs EEG electrodes to acquire EEG signals and ECG electrodes to acquire heart beat sequence. In this embodiment, similar to the embodiment in FIG. 1, EEG electrodes contacts the sampling points on the head via the head-worn structure, and additionally, two ECG electrodes are added, wherein, as shown, one ECG electrode 181 is located on the finger through a finger-worn structure and the other ECG electrode contacts the skin of head through the head-worn structure, so as to achieve an ECG signal acquisition loop. Accordingly, there is no need for the user to apply additional force on the electrode and the ECG signals can be acquired easily, thereby obtaining heart beat sequence. Alternatively, other than being carried by the finger-worn structure, the ECG electrode also can be implemented to contact other skin portion. For example, it can be carried by a wrist-worn structure 182, as shown in FIG. 4B, to contact the skin near the wrist, or by an arm-worn structure 183, as shown in FIG. 4C, to contact the skin of upper arm or front arm, or by a neck-worn structure 184, as shown in FIG. 4D, to contact the skin near the junction between the neck and the shoulder or the skin of the torso. Therefore, it can be located at any position where is capable of achieving the ECG signal acquisition loop without limitation.

When the ECG electrode is positioned at the neck, the shoulder or the back, it is preferable to adopt a wearable structure which owns flexibility at where the electrode is, so as to stabilize the contact between the ECG electrode and the skin. For example, it can be implemented to utilize materials like elastic metal, conductive rubber, conductive fiber, and/or conductive foam, for conforming to the curve of neck and should as much as possible, thereby facilitating the acquisition of stable ECG signals.

In a preferred embodiment, one of the EEG electrodes can be used to be the ECG electrode which should be mounted in the head-worn structure. Namely, one of the EEG electrodes on the head-worn structure to contact the skin is at the same time used to be EEG electrode and ECG electrode. Accordingly, expect that the manufacturing cost and complexity can be reduced, the number of contact positions also can be decreased to improve the installation convenience.

Alternatively, as shown in FIG. 5A, it also can be implemented to set two ECG electrodes on the head-worn structure. In this case, one of the ECG electrodes is located at a position capable of contacting the skin of head, and the other ECG electrode 18 is at least located at where the head-worn structure does not contact the skin of head, and thus, the ECG signal acquisition loop can be achieved by the upper limb of the user contacting the electrode 18. Accordingly, the start of the acquisition of ECG signals will be depended on the user's requirements, for example, when there is the need, the user only has to use his/her upper limb to contact the exposed electrode 18, and the measurement can be started, which is very convenient.

Moreover, the ECG electrode also can be mounted on the ear-worn structure, as shown in FIG. 5B. For example, one of the ECG electrodes can be mounted at the inner side of the ear-worn structure and the other ECG electrode 18 can be mounted on the exposed surface of the ear-worn structure. Accordingly, preferably, the ear-worn structure also can be implemented to be detachable, so that the user doesn't need to wear the ear-worn structure all the time and can connect back the ear-worn structure only when there is the need to measure ECG signals. Alternatively, if it is the situation that one of the EEG electrodes is mounted on the ear via the ear-worn structure, the ECG electrode can be also positioned in the ear-worn structure, and further, the EEG electrode and the ECG electrode also can be implemented to be single one electrode. Alternatively, it also can be implemented to be one ECG electrode carried by the head-worn structure to contact the skin of the head, and the other ECG electrode to locate at the outer surface of the ear-worn structure for being contacted by the user during measurement. Consequently, there are many different combinations and possibilities without limitation. In addition, the type of ear-worn structure also has no limitation, such as, ear clamp, ear plug or ear hook, which are all very common.

Furthermore, it also can be implemented to have optical sensor and ECG electrodes at the same time. For example, it can be similar to the embodiment of FIG. 5B, but, in the ear-worn structure, to set the optical sensor and one electrode for EEG and ECG measurement at the same time, cooperating with another EEG electrode on the head-worn structure and another ECG electrode 18 at the outer surface of the ear-worn structure. Alternatively, it also can be implemented to mount the optical sensor and the ECG electrodes on the ear-worn structure, and the EEG electrodes are both mounted on the head-worn structure to contact the skin of the head. There are many possibilities.

The advantage of this arrangement is that the cooperation of the optical sensor to acquire heart beat sequence and the ECG electrodes to acquire ECG signals can achieve a correct determination of arrhythmia syndrome conveniently. Because the optical sensor can acquire pulse variations continuously during wearing, through analyzing the continuous pulse variations, it will be able to reveal the corresponding heart beats, and thus pre-screen if there exists any possible arrhythmia event, such as, premature beats, AF (Atrial Fibrillation), tachycardia, bradycardia, and pause. However, since the analysis is based on the continuous pulse variations, some arrhythmia syndromes which must be determined after observing the ECG waveform might not be recognized, for example, premature contractions are separated into two types, one is PAC (Premature atrial contractions) occurred at atrium, and the other is PVC (Premature ventricular contractions) occurred at ventricle, and for distinguishing these two, it will need to observe if there is any abnormal shape of P wave and/or QRS complex, so as to know that the contraction is from atria or from ventricles. Since pulse is the beat of the heart as felt through the walls of a peripheral artery, it is not identical to heart beat and the accuracy thereof also can not be compared with electrocardiogram.

Therefore, through this design, if the possible arrhythmia events occurs as analyzing continuous pulse variations, it only needs to notify the user the occurrence of possible arrhythmia event, and accordingly, the user can easily and naturally start to measure ECG signals through touching the exposed ECG electrode by hand, or through wearing the wearable structure which carries the ECG electrode, e.g., on the finger, on the wrist, or on other body portion, thereby acquiring real-time electrocardiograms of possible arrhythmia events. In this case, it will be able to accurately determine whether the arrhythmia occurs or not, and even can determine the type of arrhythmia.

It should be noted that although, in figures described above, the main unit is depicted to be mainly carried by the head-worn structure, it also can be implemented to be different types. For example, the physiological signal acquisition circuitry can be directly accommodated in the head-worn structure for eliminating the main unit, such as, the head-worn structure can be implemented to have space inside or implemented to be flexible circuit board for mounting the circuitry. There are many possibilities without limitation.

Particularly, it also can use a glasses structure to achieve the contact with the sampling positions surrounding the skullcap, on the ear and/or near the ear. Namely, in all the embodiments described in FIGS. 1-2 and FIGS. 4-5, the head band can be replaced by the glasses structure.

Generally, when a glasses is worn on the head, it will naturally contact many positions, for example, but not limited, the nose pad will contact the nasal bridge, the root of nose, and/or the area between two eyes, the front portions of the glasses temples will contact the area around the temple, the rear portions of the glasses temples will contact V shaped valleys between the head and the sear, and the end portions of the glasses temples which are located behind the ears will contact the concave sides of the auricles. And, all these positions can be used to mount the optical sensor and/or electrode. Accordingly, the glasses type monitoring device will be no different from a normal glasses structure, and thus, the monitoring can be bent into the daily life even more, so as to increase the usage intention.

Here, the glasses structure refers to a wearable structure which is supported by the auricle and the nose and will contact the skin of the head and/or the ear. Therefore, it is not limited to the general glasses and also can include its transformation, for example, it can be a structure which provides opposite forces at the two sides of the head; or it also can have a asymmetric structure, such as, one temple is implemented to have bent portion at the end and the other temple is implemented to be straight; or it also can be a glasses without lens. Therefore, there are many kinds of possibilities without limitation.

As to the material of glasses, it also can have different choices. Other than the general hard material, it is also preferable to employ flexible material so as to increase the stability of electrode contact and further make the user feel more comfortable. For example, it can employ memory metal or flexible plastic to form the frame; and/or it also can be implemented to mount elastic rubber or silicon at the position of electrode, so as to stabilize the contact thereof.

Further, the combination manner of the optical sensor, the EEG electrode and/or the ECG electrode with the glasses structure also can have many possibilities.

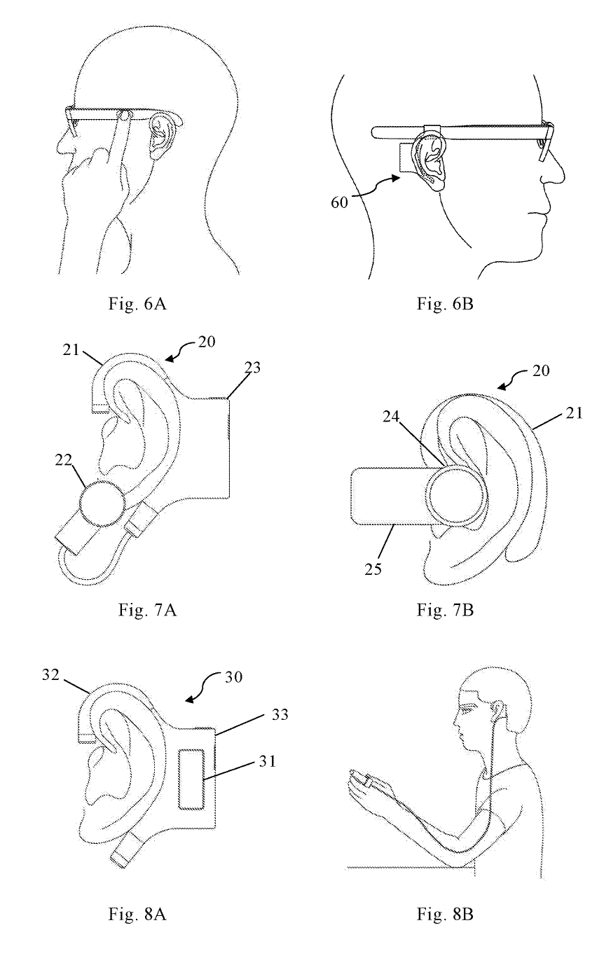

It should noted that as mentioned above, only one of the at least two ECG electrodes will contact the head and/or the ear via the glasses structure, and another ECG electrode is positioned at an exposed surface of the glasses structure to being touched by the user's hand for acquiring ECG signals, as shown in FIG. 6A, or alternatively, another ECG electrode is carried by another wearable structure to position on another body portion of the user, such as, the neck, the shoulder, the back, the arm, the wrist, the finger and the chest etc. Thus, in the following, the descriptions about the combination of the optical sensor/electrode with the glasses structure are focused on at least two EEG electrodes, or at least one optical sensor, or at least one ECG electrodes.

For example, the optical sensor/electrode and the circuitry (such as, processor, battery, wireless transmission module) can be directly embedded in the glasses structure, e.g., glasses temple, and/or lens frame, so that the positioning of the optical sensor/electrode can be achieved directly through the operation of wearing the glasses structure. Alternatively, it also can employ an additional structure to achieve the arrangement of the optical sensor/electrode and the circuitry, as shown in FIG. 6B, the additional structure 60 is implemented to extend from one glasses temple, so as to contact, e.g., two EEG electrodes, one ECG electrode, and/or the optical sensor, with the skin near the ear at the same side. Or, the additional structure also can be implemented to be two and extend from two glasses temples, wherein each has one electrode mounted thereon for acquiring EEG signals through respectively contacting the skin near both ears, and the ECG electrode and/or the optical sensor can be located on any one without limitation. In this case, the electrical connection between two additional structures can be configured to pass through the glasses structure, and the circuitry can be configured to be partial or all arranged in the glasses structure and/or the additional structures. Further, the additional structure also can be configured to be detachable, so that the user can selectively attach the additional structure to the glasses structure only when wanting to do the measurement. There is no limitation.

Moreover, the wearable physiological monitoring device of the present invention also can be carried by an ear-worn structure for being installed on the user's ear. For example, FIGS. 7A-7B show exemplary embodiments of ear-worn physiological monitoring device 20 with EEG electrodes and optical sensor. In the embodiment of FIG. 7A, the ear worn structure is implemented to be an ear-hook structure 21 with an ear-clamp structure 33, wherein the ear-clamp structure 22 is mounted on the ear lobe for positioning the optical sensor and the reference EEG electrode, and the active EEG electrode is mounted on the ear-hook structure 21 or other portion of the ear-worn structure, e.g., the housing 23, capable of contacting the skin of the ear or around the ear for achieving EEG signal acquisition, namely, a position capable of detecting the activity of cortex. In the embodiment of FIG. 7B, the ear-worn structure is implemented to be an ear-hook structure 21 with an ear-plug structure 24, wherein the optical sensor and the reference EEG electrode are mounted on the ear-plug structure to contact the canal, the opening of canal, and/or the inferior concha, and the active electrode is located on the ear-hook structure 21 or the other portion of the ear-worn structure, e.g., the housing 25, for contacting a position capable of acquiring EEG signals on the ear or near the ear. Therefore, there are many kinds of possibilities. Besides, it also can be implemented to use single ear-worn structure, namely, only one ear-hook, one ear-clamp or one ear-plug, to carry the EEG electrodes and the optical sensor, without limitation.

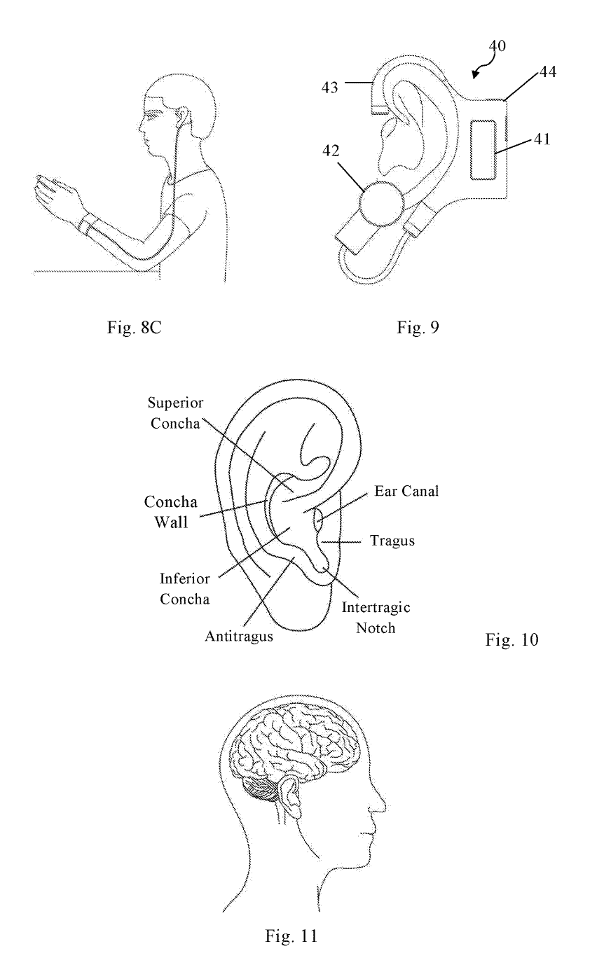

Moreover, as shown in FIG. 8A, the ear-worn physiological monitoring device 30 is configured to have EEG electrodes with ECG electrode. In this embodiment, one ECG electrode 31 is configured to be exposed for being contacted by an upper limb of the user to achieve the ECG signal acquisition loop. The other ECG electrode is configured to contact the skin of the ear or around the ear through the ear-worn structure, and it can be implemented to be shared with one of the EEG electrodes or to set independently without limitation. EEG electrodes are configured to contact positions capable of acquiring EEG signals, namely, capable of detecting the activity of cortex, on the ear or near the ear via the ear-hook structure 32 and/or the housing 33. Alternatively, it also can be configured to additional have an ear-clamp structure, such as, to clamp on the lobe or the helix, for mounting one shared EEG and ECG electrode inside and one ECG electrode 31 on the outer surface, and the active EEG electrode can be mounted on the ear-hook structure.

Besides, the ECG electrode for contacting the upper limb also can be configured to mount on the finger through a finger-worn structure, as shown in FIG. 8B, or on the wrist, on the arm, or one neck, shoulder or back. FIG. 7C shows the situation of using the neck-worn structure to contact the electrode with the skin of the neck, the shoulder or the back, which also provide operation convenience. Of course, the electrode also can be selected to contact other body portion, such as, the torso, without limitation.

Similarly, in another embodiment, as shown in FIG. 9, the ear-worn physiological monitoring device 40 is configured to have EEG electrodes, optical sensor and ECG electrodes. The optical sensor is mounted on the lobe through an ear clamp 42, one ECG electrode 41 is exposed for being contacted by an upper limb, and the other ECG electrode can be implemented to locate inside the ear clamp 42 or on the ear hook 43 and/or the housing 44 to contact the ear or the area near the ear. Further, as mentioned above, EEG electrodes can have different possibilities. For example, it can be that the reference electrode is mounted on the ear clamp 42 or further to share with the ECG electrode, or it also can be that two EEG electrodes' contact with the skin are both achieved by the ear clamp and/or the housing. There is no limitation.

Followings are the explanation of some special positions on the auricle for locating electrode in the present invention. Please refer to FIG. 10 which shows the structure of auricle (also named as pinna). Within the concave side, around the superior concha and the inferior concha, there is a perpendicular area which is extended from the concha floor (namely, a surface parallel to the scalp) up to the antihelix and antitragus, and called as concha wall. This naturally physiological structure just provides a continuous surface perpendicular to the concha floor. And, the intertragic notch which is adjacent to the antitragus and the tragus also provide a contact area perpendicular to the concha floor.

In experiments, it found that the continuous perpendicular area consisting of the concha wall, the antitragus, the intertragic notch and the tragus are positions where can acquire EEG signals with sufficient signal strength for signal analysis and brain activity information provision. Further, when the contact of electrode is aimed at this continuous perpendicular area, the direction of force for fixing the electrode will be parallel to the concha floor. Particularly, when it is implemented as an ear plug, the against force between the ear plug and the structures at the concave side of auricle will naturally achieve a stable contact between the electrode and this perpendicular area.

Besides, it also found that the convex side of the auricle is also a position where can acquire EEG signals with sufficient signal strength. And, this contact position is suitable for ear-hook structure or glasses structure. Generally, the ear-hook structure includes one part at the concave side and another part at the convex side which have mutual forces toward each other for fixing the structure on the ear. Therefore, if the electrode is selected to contact the convex side, it will just match to the directions of the forces provided by two parts of the ear hook structure, and thus, the contact of electrode can be achieved naturally and stably.

When adopting the glasses structure, the V shaped valley between the auricle and the head and/or the upper portion of the convex side will just be the contact area of the glasses temple. And, if the end of the glasses temple can be implemented to bend more, it will be able to contact the lower portion of the convex side. Therefore, the contact also can be achieved naturally.

Then, please refer to FIG. 11 which is a schematic view of the positions of the cortex, which is inside the head, and the auricle. As shown, the cortex is located at the upper portion inside the head and the auricles are located at two sides of the head in an outwardly protruding manner. Generally, take the ear canal as the partition, the upper portion auricle is right aside the cortex, and the inside portion of the head corresponding to the lower portion auricle does not include the cortex.

The experiments further found that, on the auricle, it can acquire EEG signals with good quality at the upper portion, and the lower the acquisition position, the weaker the EEG signal. After observing the physiological structure of the head, the inference is because the upper portion of auricle just corresponds to the cortex, through the transmission by the head bone and the ear bone, the EEG signal can be detected at the upper portion of auricle. As to the lower portion of auricle, because it is farther from the cortex and the canal also provides the separation effect, the signals become weaker as the position goes lower of the auricle. Accordingly, in the present invention, when the auricle (the concave side and the convex side) is used to acquire EEG signals, in principle, take the ear canal as partition, the upper portion auricle is regarded as where capable of acquiring EEG signals and is suitable for mounting active electrode, and the lower portion auricle is regarded to have weak EEG signals and is suitable for mounting reference electrode.

It should be noted that when adopting the ear-worn type, the physiological signal acquisition circuitry, as shown in FIGS. 6-8, can be accommodated in the housing carried by the ear-worn structure, or separately in the ear-worn structure and the housing, without limitation. Alternatively, the housing also can be eliminated, so that the circuitry can be directly accommodated in the ear-worn structure, such as, in the ear-hook structure, the ear-plug structure, and/or the ear-clamp structure. There are many possibilities. Besides, the ear-worn structure also can be implemented to be only one kind or to combine plural structures. For example, the ear-clamp structure, the ear-hook structure and the ear-plug structure can be used individually or combined two or three together. It can be changed depending on the real situation.

In a preferred embodiment, the magnetic force is used to attach the electrode(s) and/or the optical sensor onto the ear. For example, it can be two elements which magnetically attract across the ear, and the electrode and/ or optical sensor can be implemented to locate on two or one of the elements. Here, the two elements can be implemented to be both magnetic, for example, by inserting magnetic material into the element or by making the element directly of magnetic material; or to be made of material which is attracted by magnetism, for example, it can be one element is magnetic and the other is attractable by magnetism or both elements are magnetic, without limitation.

In another preferred embodiment, a movement sensing element, such as, accelerator, can be further included in the device for detecting the movement of the user during the measurement, for example, the movement of body, the head, and/or the ear. Accordingly, it will be able to perform a correction for the acquired physiological signals, such as, the EEG signals, the ECG signals and/or the optical signals, so as to correct the instability caused by the movement of head or body. Therefore, the provided information will be more close to the real situation, which is beneficial for improving the effect achieved through neurofeedback.

Particularly, it also can be further implemented to combine the glasses structure with the ear-worn structure for carrying the electrodes and/or the optical sensor. For example, an ear plug or ear clamp can be configured to extend from the glasses structure, or the glasses structure can be configured to include a port for electrically connecting an ear plug or ear clamp. Accordingly, there will be many possibilities. For example, in the embodiment of EEG electrodes with optical sensor, the electrode on the glasses structure can be configured to contact the V shaped valley, the convex side, the temple, the nose bridge, the root of nose, and/or the area between two eyes, and the electrode on the ear plug structure can be configured to contact the concha wall, the intertragic notch and/or the tragus for acquiring EEG signals, and the optical sensor can be selected to locate on the glasses structure or the ear-worn structure. Alternatively, it also can be implemented to mount two EEG electrodes on the glasses structure and the optical sensor on the ear-worn structure. In the embodiment of EEG electrodes with ECG electrodes, the exposed ECG electrode can be selected to locate on the glasses structure or the ear-worn structure for cooperating with the ECG electrode at the inner side of the glasses structure, and further, if the ear-worn structure is connected via the connecting port, the user can connect the ear-worn structure back only when there is the need to detect ECG signals. Besides, the ear-worn structure also can be implemented to have the optical sensor. Thus, there are many kinds of possibilities.

Except for the EEG electrode mounted on the ear-worn structure, the head-worn structure, and/or the glasses structure, it also can be configured to include other EEG electrode. For example, it can be implemented to have electrode(s) extended from the ear-worn structure, the head-worn structure or the glasses structure for locating at other portion of the head, such as, at the forehead to acquire EEG signals of frontal cortex, at the top of head to acquire EEG signals of parietal cortex, and/or at the rear of head to acquire EEG signals of occipital cortex. Particularly, if the glasses structure is employed, the locating of the electrode(s) at the rear of head also can be achieved by extending the end portion of the glasses temple. Therefore, it can be changed depending on the real situation without limitation. Further, if the location of the electrode has hairs, e.g., the top and rear of head, it can be selected to employ needle electrode or other kind of electrode capable of penetrating the hairs to acquire signals for more convenient operation.

In addition, other physiological signals also can be detected. For example, it can be configured to detect physiological signals which are usually used in biofeedback process, such as, EDA (Electrodermal Activity) and peripheral temperature which are also controlled by ANS, so as to be a basis for providing feedback information, such as, to additionally provide ANS related information other than brain activity related information, or to provide information which commonly reflects both physiological conditions. Therefore, it only needs to be correct and effective information capable of representing the real time physiological condition, without limitation.

As known, the blood pressure level and the ANS activity have particular relationship, generally, the increase of SNS activity will cause the blood pressure level to raise, so that through cooperating ECG electrodes with optical sensor, it will be able to obtain PTT (Pulse Transit Time). Then, through the particular relationship between PTT and blood pressure, the reference blood pressure can be calculated. As a result, it will be able to provide the user the blood pressure variation trend during the nurofeedback process, or the blood pressure difference before and after the neurofeedback section, so as to help the user to know the influence of neurofeedback process on the blood pressure. Similarly, it also can be configured to mount two optical sensors, such as, one on the head/ear, and the other on the finger, so as to obtain blood pressure information through calculating the pulse wave velocity time difference of two locations.

Moreover, in the present invention, the brain activity information and the breathing guiding signal are implemented to provide to the user through a perceptible signal producing source. Through a communication between the perceptible signal producing source and the wearable physiological monitoring device, such as, through the general wireless communication, e.g., Bluetooth, WiFi, the perceptible signal producing source can receive an input from the device and then provide thereof to the user in real time, so as to achieve the neurofeedback loop.

Here, the perceptible signal producing source can use visually perceptible signal, auditory perceptible signal and/or tactile perceptible signal to provide the user the brain activity related information and the breathing guiding signal, for example, through the variations of luminous color, luminous intensity, sound, voice, and/or vibration, without limitation. And, the perceptible signal producing source also can be implemented to various types. For example, the perceptible signal producing source can be particularly implemented as an independent lighting object, such as, a ball object or an object of any shape, or can be implemented to be a device with display and/or sounding function, such as, a mobile phone, a watch, a tablet, and a personal computer, or can be implemented to be a device worn on the user's body with display, sounding, and/or vibration function, such as, single ear headset, dual ear headset, or glasses.

Alternatively, the perceptible signal producing source also can be implemented to be the display unit, the audio module, and/or the vibration module combined with the wearable physiological monitoring device. For example, no matter the head-worn structure or the ear-worn structure is employed, the perceptible signal producing source can be implemented to be a display element, a luminous source, and/or a headset extended therefrom. For example, it can be implemented to be a glasses for carrying EEG electrodes and the heart rate sensing unit and utilizing the lens to display the information, e.g., the light can be guided to the lens for showing the color variations, the lens can be implemented to own display function, and/or the glasses can be implemented to combine a headset near the glasses temple for providing sound and/or voice. Alternatively, it also can be implemented to be a headset for providing sound and/or voice while carrying EEG electrodes and the heart rate sensing unit, and/or a display element or luminous source can be extended from the headset for providing visually perceptible signal. Further, any position of the device where contacts the skin can be configured to provide vibration, such as, the portion of the glasses temple that contacts the user's temple, or the headset. Therefore, there is no limitation.

When the user employs the wearable physiological monitoring device of the present invention to perform a nurofeedback process, as shown in FIG. 1, he/she can wear the device on the head for, through the EEG electrodes and the optical sensor at the inner side of the head-worn structure, respectively acquiring EEG signals and heart beat sequence, then put the perceptible signal producing source which is implemented as the lighting object in front of him/her, and perform the communication between the device and the lighting object, so as to ready for the neurofeedback process.

Because the process combines the breathing training and neurofeedback, as mentioned above, for performing the breathing training, it will need to provide the user the breathing guiding signal, and for performing the neurofeedback, it will need to provide the user the information of physiological activity which changes corresponding to the neurofeedback. And, here, the light object is the providing medium for both.

In this embodiment, the perceptible signal produced by the light object includes two factors, luminous intensity and luminous color, wherein the luminous intensity is used to represent the breathing guiding signal, and the luminous color is used to represent the brain activity related information.

Because the purpose of the breathing guiding signal is to be followed by the user to inhale and exhale, it will need to represent the difference between the inspiration and the expiration. And, the continuous strong and weak variation of the luminous intensity of the lighting object is suitable for representing the difference between the inspiration and the expiration, such as, the gradually increased luminous intensity can be used to guide the user to gradually inhale, and the gradually decreased luminous intensity can be used to guide the user to exhale gradually. As a result, the user can clearly and easily understand and follow the indications.

When the target of neurofeedback process is relaxation, one selection is to observe the a wave percentage in brainwave. Generally, dominant a wave can be observed in awake and relaxed individuals. Accordingly, after the start of neurofeedback process, the lighting object provides the breathing guiding signal (through a continuous variation of luminous intensity) for guiding the respiration of the user, and at the same time, the physiological monitoring device worn on the head acquires and analyzes the brainwave to obtain an analysis result, such as, the percentage of a wave, and based on the analysis result, a brain activity related information is generated. Then, the light object changes the luminous color thereof in accordance with the brain activity related information in real time.

For example, it can be implemented to acquire a reference value, such as, the a wave percentage in brainwave, right at the beginning of the process, and then compare the analysis result with the reference value to know the difference, such as, the percentage increases or decreases, so that, based on this, the lighting object can change the luminous color thereof in real time for conveying the change of physical condition to the user. For example, the change can be achieved by employing multiple colors, such as, use blue color to represent a more relaxed physical condition, and red color to represent a more nervous physical condition; or alternatively, also can be achieved by different shades of one color, such as, the darker shade represents more nervous, and the lighter shade represents more relaxed. As a result, the user can simply understand his/her mental and/or physical condition through the color variations, so as to perform the self-regulation while following the breathing guiding, thereby changing the luminous color toward the relaxation target.

Alternatively, the relaxation degree or emotional state of individuals also can be revealed through observing the energy balance and/or the synchronization among different brain portions. For example, when people have positive emotion, the left prefrontal cortex will be activated, and when negative emotion is raised, the right prefrontal cortex will be activated. Therefore, through detecting the EEG signals at these two brain portions, such as, at Fp1 and Fp2, it will be able to understand the activities thereof. Other studies also shown that when the brain is in a a wave synchronization state, the individual will have a clear consciousness but in a relaxed state. Therefore, through detecting the activities of different brain portions, such as, at Fp1 and Fp2 for detecting prefrontal cortex, at C3 and C4 for detecting parietal cortex, at O1 and O2 for detecting occipital cortex, and at T3 and T4 for detecting temporal cortex, it will be able to understand the synchronization condition. In this case, it will only need to adjust the positions of EEG electrodes in the head-worn structure or to utilize two ear-worn structures to locate the electrodes respectively on the ears, and then the activities of different brain portions can be obtained.

When the target of neurofeedback is to relax, the ANS activity that can be obtained by analyzing the heart beat sequence also can be the basis for adjusting the luminous color, for example, when the PSNS activity increases and/or the ratio of PSNS/SNS increases, it represents the individual is in a relaxed state, so that the evaluation of relaxation can be performed to combine this information with the brain activity related information, for being the basis to adjust the feedback luminous color.

Moreover, because the heart beat sequence also can be used to obtain RSA information, it will be able to employ the synchronization among heart rate, respiration and EEG signals as the feedback information. According to studies, inspiration and expiration will cause the fluctuations of blood in the vessels, and the fluctuations will arrive brain with the blood flow, so as to cause fluctuations of brainwaves in low frequency section, e.g., lower than 0.5 Hz. Accordingly, except for knowing if brainwave and respiration have in the synchronization state due to resonance, it also can reveal the breathing pattern via observing brainwaves. Furthermore, the sinus node of heart and the vascular system are also controlled by SNS system, and SNS system also will feedback the changes of heart rate and blood pressure to the brain via baroreceptor system and thus influence the function and operation of brain, for example, to influence cortex, which can be detected via electroencephalograms. In addition, a consciously controlled breathing can cause the change in heart rate due to its influence on ANS. Therefore, these three physiological factors do influence each other, and a great synchronization thereamong represents the individual is under a relaxed state. Accordingly, the analysis result of a correlation among these three physiological signals, such as, the above mentioned synchronization, also can be used as the feedback information for the user to perform the self-regulation in the neurofeedback process.

Besides, the breathing pattern of the user also can be revealed by observing the fluctuations of blood flow, for example, through the optical sensor positioned on the ear or forehead to acquire pulse variations, the variation of blood flow will be revealed.

When the target is to improve concentration, it can select to observe the ratio of θ wave and β wave. Dominant β wave represents individuals in an awake and focused state, and dominant θ wave represents individuals in an idling and drowsiness state. Therefore, through increasing the percentage of β wave, it will be able to achieve the purpose of improving concentration. For example, one of the methods for curing ADHD (Attention Deficit Hyperactivity Disorder) patients is to perform a neurofeedback by observing the ratio of θ/β. Accordingly, after employing the system of the present invention to perform the neurofeedback process, the lighting object provides breathing guiding signal (through continuous variation of luminous intensity) to guide the user to adjust the breathing, and at the same time, the physiological monitoring device worn on the head detects EEG signals and analyze the percentages of θ wave and β wave, such as, respective θ wave percentage and β wave percentage related to total brainwave, or the value of θ/θ+β and β/θ+β. Then, in accordance with the analysis result, a brain activity related information is generated, and based on it, the light object produces the real time variations of luminous color for showing the user the brain activity change, for example, the change can be achieved by employing multiple colors, such as, use blue color to represent a low concentration state, and red color to represent a highly concentrated state; or alternatively, also can be achieved by different shades of one color, such as, the darker shade represents more concentrated, and the lighter shade represents low concentration. As a result, the user can simply understand his/her concentration state through the color variations, so as to perform the self-regulation while following the breathing guiding, thereby changing the luminous color toward the concentration target.

Other than observing θ wave and β wave, SCP (Slow Cortical Potential) is also a factor that can be observed and used in neurofeedback process for improving concentration. For example, when curing ADHD patients, the negative shift of SCP is related to more focused and concentrated, and the positive shift of SCP is oppositely related to reduced concentration.

Here, the brain activity displayed by the luminous color can be different. For example, as mentioned above, it can be the variation of the calculated relaxation or concentration degree, or it also can be the variation of physiological signals, such as, the a wave percentage, without limitation. Further, the changing manner of the luminous color also has no limitation. The point is to let the user understand his/her physical condition in a simple and clear way, so as to trigger the user to perform self-regulation and achieve the target physiological state.

Therefore, through the device of the present invention, the user can combine the breathing adjustment and the self-regulation process, which influences brain function, in a natural way without particular learning. The key point is the perceptible signal produced by the perceptible signal producing source includes two information, such as the embodiment of FIG. 1, the single lighting object produces one visually perceptible signal which employs luminous intensity and luminous color to respectively represent breathing guiding signal and real time physical condition.

In the present technologies, the general way to provide feedback to the user in the neurofeedback section is, for example, an object which will move according to the physiological state, such as, a flowing ball will fly higher when the user becomes more relaxed; or a figure which will change the shape, such as, a flower will bloom more when the user becomes more relaxed; or to show the value directly. As to the breathing guiding method, generally, it will, for example, employ the ups and downs of waveform to represent inspiration and expiration. Accordingly, the combination of these two will become too complicated for the user to understand, and also, the various visual changes also might influence the focus of the user, thereby oppositely increasing the mental stress and thus reducing the effect of neurofeedback.

Therefore, in view of these possible problems, when considering how to provide the user the information, the inventor selects a method that provides two kinds of information in only one single object, thereby simplifying as far as possible, making the operation easier and thus reducing the mental stress as performing the neurofeedback process. The advantages of the present display method include:

1. The continuous changes of luminous intensity are similar to the usual way to express rhythm, so that there is no need for the user to re-learn the meaning and can follow the guiding intuitively.

2. The luminous color is easier for the user to understand the physical condition, as compared with the directly-shown values. And, it is also easy to develop a sense of identity by using different colors or the shade of color to represent the change of degree or level. Accordingly, the user can respond to perform the self-regulation more naturally.

3. The visual focus is reduced to only one even there are two processes combined, which facilitates the user to concentrate even more.

Therefore, through the well-designed expression method of the perceptible signal, the possible complexity from combining two processes can be eliminated, which not only effectively reduces the user's burden, but also provides a novel feedback process with doubled effects.

Other than using the single light object to provide variations of the luminous intensity and the luminous color, it also can employ other element/unit/device with display function to achieve this. For example, it can be a lighting source on the screen, such as, the screen of the tablet, the mobile phone, the watch, or the personal computer; and further, the lighting source also can be implemented to be a portion of a figure, such as, to locate at the head or the belly of a human, so as to help the user to imagine the activity inside the body while performing the self-regulation. Further, except the solid lighting source, it is also preferred to employ a lighting circle. For example, a light circle around the head also can help the imagination. Besides, the diameter of the lighting source or lighting circle also can be implemented to change along with the variation of luminous intensity, so as to enhance the guiding effect. Therefore, there is no limitation and can be varied depending on the practical situation.

Moreover, an additional auditory perceptible signal also can be provided, such as, sound and/or voice, so as to provide another choice when the user wants to close the eyes in the neurofeedback section. For example, the intensity change of volume can be used to represent the continuous variations of inspiration and expiration, and different kinds of sounds, such as, birdcalls and the sound of waves, or different music tracks can represent different physiological states. Alternatively, it also can be implemented to guide the user to inhale or exhale through voice, and then use the frequency of sound to represent the physical condition, such as, higher frequency corresponds to more nervous, and lower frequency corresponds to more relax. Thus, there is no limitation. And, the auditory perceptible signal can be produced by the perceptible signal producing source and/or the wearable physiological monitoring device, without limitation.

Identically, the breathing guiding signal also can be implemented to have many different possibilities. In the general breathing training process, the breathing guiding signal can mainly be divided into three types. One is to provide a preset and fixed respiratory pattern, such as, the respiratory rate is set and fixed to be 8 times per minute. Another is to provide a respiratory pattern which will change along with time, for example, a 15-minute breathing guiding signal can be divided into three sections, wherein the respiratory rate is set to be 10 times per minute in the first 5-minute section, 8 times per minutes in the middle 5-minute section, and 6 times per minute in the last 5-minute section. The other is to provide a respiratory pattern which will dynamically change in accordance with the physiological state. And, in the present invention, the breathing guiding signal can be implemented to be any kind above. For example, through the EEG signals and/or heart beat sequence acquired by the wearable physiological monitoring device, the breathing guiding signal can be implemented to dynamically change with the physiological state, so as to more effectively guide the user to achieve the target physical condition.

As to how the physiological state influences the breathing guiding signal, it also has many possibilities. For example, it can be implemented to obtain the user's real respiratory behavior through analyzing the heart beat sequence, so as to realize the difference from the guiding signal, thereby being the basis to adjust the breathing guiding signal, such as, if the user's real respiratory rate is already lower than the guiding signal, then it will be able to reduce the respiratory rate of the breathing guiding signal for further increasing the biofeedback effect.

Alternatively, the heart beat sequence also can be analyzed to obtain the ANS activity, and thus reveal the relaxation degree of the user. Therefore, if it found that the relaxation degree has increased and stay in a stable level, the respiratory rate of the guiding signal can be then lowered down, such as, from 8-10 times per minute to 6-8 times per minute, for further increasing the relaxation degree. Alternatively, it also can be implemented to stop the breathing guiding when the user's relaxation degree has reached the preset target or the respiration has matched the guiding signal and also very stable, so as to help the user to focus more on the self-regulation process, and only when it finds the user's respiration becomes unstable or the relaxation degree has changed, the breathing guiding can start again. Therefore, there is no limitation.

Particularly, it also can be implemented to intermittently provide and not provide the breathing guiding signal, so that the user can alternately perform the breathing training process and the self-regulation process. According to studies, the effect of biofeedback can be doubled if the user's breathe is in a smooth and stable state. Therefore, through firstly providing the breathing guiding signal for a period of time to make the user being used to the provided respiratory pattern and become stable, and then stop the provision of breathing guiding signal, the user will be able to naturally remain the respiratory pattern and purely focus on the self-regulation process. And, this procedure will be able to further improve the effect of biofeedback.

And, because ANS has a delayed response to the breathing training, through this intermittent provision of breathing guiding signal cooperating with the combined breathing training plus self regulation process of the present invention, in the period of not providing breathing guiding, not only the influence of breathing training on ANS can be shown, the user also can conveniently perform the self-regulation process, thereby enhancing the effect of breathing training.

The alternation of breathing training and self-regulation, namely, the provision of the breathing guiding signal, can be decided by the user's physiological state, or can be based on a fixed interval, without limitation. In addition, the alternation also can be implemented to have different respiratory rates, such as, to alternate between 6-8 times per minute and 10-12 times per minute, so as to facilitate, for example, a conversion training of focus, thereby achieving a more flexible controlling ability.

It should be noted that the provision of the breathing guiding signal (whose respiratory pattern can be preset fixed, changed along with time, or dynamically altered in accordance with physiological state) can be implemented that the wearable physiological monitoring device generates and transmits it to the perceptible signal providing source, e.g., smart phone, tablet, smart watch, and then, the perceptible signal providing source provides it to the user. Alternatively, it also can be the perceptible signal providing source originally includes a preset respiratory pattern for providing to the user, and will further receive the input from the wearable physiological monitoring device for being the basis to adjust the breathing guiding signal. There is no limitation.

In another aspect of the present invention, it also can be implemented to employ an auditory perceptible signal to provide the brain activity related information and the breathing guiding signal. As shown in FIG. 2, the user can perform the breathing training and self-regulation in accordance with the audio breathing guiding signal and the audio brain activity related information provided by the mobile phone.

The method of auditory perceptible signal to express respiratory pattern can include, but not limited, for example, to utilize the interval of sound to represent the starts of inspiration and expiration, or to utilize the change of sound frequency or volume to represent the continuous variations of inspiration and expiration, or to employ different kinds of sounds to represent inspiration and expiration, such as, different music tracks, or to employ a sound file with periodic change, such as, the sound of waves, or to guide the user to inhale and exhale by voice, such as, it can pronouns “inhale” and “exhale” at the points for inhaling and exhaling.

When the auditory perceptible signal is at the same time used to represent the biofeedback information, it also has many possibilities. For example, a gradual variation of sound frequency or volume can be used to represent the approaching or getting away from the target. Alternatively, it also can be implemented to use particular kind of sound or music track to represent particular information, such as, the target is achieved or not yet achieved. Alternatively, it also can inform the user via voice if the target is approaching. Therefore, it only needs to be distinguished from the breathing guiding signal, without limitation.

When the target is to relax, in one embodiment, the interval beep sound can be used to guide the user to inhale and exhale, and the sound frequency can be used to represent the relaxation degree, such as, the higher the sound frequency, the more nervous the user is, and the lower the sound frequency, the more relaxed the user is. Accordingly, when the user heard a high frequency beep sound, he/she will know that the physiological state thereof is too nervous, and should try to relax while following the guiding to inhale and exhale, Therefore, even only employing one single sound signal, it is still able to let the user know two information.

In another embodiment, the strength of sound volume is used to represent the continuous variations of inspiration and expiration, and the type of sound is used to represent the relaxation degree, for example, the birdcall represents a more nervous state, and the sound of wave represents a more relaxed state. It also can be clearly expressed.

The auditory perceptible signal can be produced by the audio module combined with the wearable physiological monitoring device. For example, it can be a headset combined with the head-worn or hear-worn physiological monitoring device, so that only through wearing one single device, the user can simultaneously have the information of biofeedback and breathing guiding, which provides highly mobility and convenience. Further, if the whole device is implemented to be glasses type or ear-worn type, it will be even more aesthetic and bent into daily life, especially suitable for performing eye-closed feedback section in the transportation period. Particularly, the adopted audio module or earphone can be not only air conduction type but also bone conduction type, for example, a bone conduction speaker can be directly mounted at a position of the glasses structure which will contact the head bone, or it also can employ a bone conduction earphone extended from the glasses structure. There is no limitation.

When the device is implemented as glasses structure, preferably, it will be able to provide the functions of earphone and/or microphone, for example, through mounting an acoustic component and/or a sound receiving element (e.g., microphone) thereon, or to have the earphone extended out. Here, particularly, the adopted acoustic component or earphone can be not only air conduction type but also bone conduction type, for example, a bone conduction speaker can be directly mounted at a position of the glasses structure which will contact the head bone, or it also can employ a bone conduction earphone extended from the glasses structure. There is no limitation.

Moreover, in another aspect of the present invention, it also can be implemented to employ a tactile perceptible signal to provide the brain activity related information and the breathing guiding signal. For example, it can employ vibration signal to notify the user the correct time points of expiration and/or inspiration; or it also can be the vibration signal only generates when the user's respiratory pattern is deviated from the preset guiding signal. And, it can use the strength of vibration to represent different physiological states, such as, the stronger the vibration, more nervous the user is, and along with the increase of relaxation degree, the vibration becomes weaker.

Advantageously, when employing the auditory and/or tactile signals, the user can close his/her eyes, so that it will facilitate the relaxation and also the adjustment of respiration.

In addition, it also can be implemented to simultaneously provide auditory perceptible signal and tactile perceptible signal. For example, the vibration signal is used to notify the user the time points of inspiration and/or expiration, and the change of physiological state is reported to the user via voices; or it also can utilize the sounds to provide the breathing guiding signal and the vibrations to notify the current physiological state, without limitation. Preferably, it can be implemented to be a headset with vibration function, and in this case, the user not only can close eyes but also will not influence people around when performing the neurofeedback section according to the present invention.

In addition, the device of the present invention also can be configured to communicate with a portable electronic device, for example, to execute wired or wireless communication via, e.g., earphone socket or Bluetooth, with a portable electronic device, such as smart phone, tablet or smart watch. Thus, when there includes the acoustic component (air conduction or bone conduction type) and the sound receiving element, the device of the present invention can be used as a hand-free handset. Further, through setting vibration module, acoustic component (air conduction or bone conduction type), display element, and/or light emitting element etc, the glasses type or ear-worn device of the present invention can be used as an information providing interface of the portable electronic device, such as, to notify the call or message. Accordingly, the device can be bent in the daily life even more. As to how the notification presents to the user, it can be in different manners, such as, sound, vibration, light, display on the lens, without limitation.

In still another aspect, the device of the present invention is also suitable for being used as a brain-computer interface (BCI) due to the wearable type thereof. Since the detected physiological signals include EEG signals and hear rate sequence, there are several ways to generate commands. For example, because the percentage of a wave will have significant change in accordance with the motions of closing and opening eyes, such as, generally, when eyes are closed, the percentage of a wave will have a significant raise, so that this can be used as the basis to generate commands. Moreover, when EEG electrodes are located around the eyes, such as, at the nasal bridge, the root of nose, the area between eyes, the temples, it will also acquire electrooculographic (EOG) signals, so that it will be able to generate commands through, e.g., blinking or rolling the eyes. Furthermore, it is also possible to control the respiration, and as mentioned above, the respiration not only will influence the heart rate (namely, the so called RSA), also will cause the fluctuation of brainwaves in low frequency section. Accordingly, under the configuration of the present invention, since the change of respiratory pattern can be obtained no matter through detecting of EEG signals or heart beat sequence, it will also be able to use to generate commands, for example, the user can on purpose elongate the expiration period, or to make a deep breath to increase HRV and thus increase the amplitude of RSA. Therefore, there is no limitation.

Besides, it can further employ a motion detecting element, such as, accelerator, to generate more commands, for example, all above mentioned physiological phenomena can further cooperate with the body motion, such as, head nodding, swing, or rotating, to obtain more combinations and thus broaden the application range, such as, this will be suitable for being applied in VR (Virtual Reality) games or smart glasses.

The neurofeedback process of the present invention also can be integrated into games. For example, the auditory and/or visual signals which will be changed according to the physiological state can be implemented to be object, human, and/or sound in the games, to provide interactions with the user for having more fun. For example, it can employ scores to represent the relaxation degree in a neurofeedback section, such as, the increase percentage of a wave, and because biofeedback have cumulative effect, the scores at different times, and different sections can be accumulated, as a result, the user will be able to know his/her overall efforts through reviewing the scores, which can help the user to find fulfillment. In this case, it also can be implemented to have different checkpoints, for example, after reaching particular checkpoint, particular function will be activated, so as to increase the challenge desire.

Another way is to provide rewards. For example, after the scores reach particular checkpoints, the user will, such as, have more choices of characters, clothes, have a halo, be equipped with more accessories or treasures, or be upgraded to a higher level. There is no limitation and all kinds of methods which are familiar in the online or mobile games can be used.

Here, different from the general games, the accumulation of biofeedback is mainly constructed on the premise of continuous usage, namely, if the performing interval between biofeedback processes becomes too long, the cumulative effect will be disappeared. Therefore, the principle of score calculation can be, for example, the accumulated scores will gradually be decreased as the time of performing interval gradually becomes longer, and if the interval becomes too long, the scores will be eliminated and the user has to start again, such as, the scores will be decreased to 75% if the user does not perform biofeedback process for two consecutive days, 50% for three consecutive days, and so on, and finally the score will be decreased to 0 after five days. Through this, it will encourage to user to use every day.

Therefore, through being applied in games, expect for making the biofeedback process more fun, the user also will feel the physiological change via the scores in real time, thereby facilitating the user to have a target and more power.

Furthermore, the device of the present invention also can be applied to acquire sleep related information. As ones skilled in the art known, EEG signals are the main source to decide sleep stage, and the traditional way is to put multiple electrodes on the head via multiple connecting wires from a machine, so that it is very inconvenient, especially during sleep, the user not only almost can not move, but also might have difficulty in falling asleep. Therefore, if the electrodes can be set simply through ear-worn structure or glasses structure, the whole process will become more natural and less burden, thereby reducing the influence on user's sleep during measurement, and thus, a result that is closer to the daily normal sleep condition can be acquired.

In addition, it also can be configured to acquire other electrical physiological signals, such as, EOG signals, electromyography (EMG) signals, electrocardiography (ECG) signals and electrodermal activity (EDA) through setting more electrodes or sharing the exited electrode. As known, all these signals are included in the polysomnography (PSG) examination. For example, EOG signals can provide the information of REM (Rapid Eye Movement), EMG signals can provide information of sleep onset and sleep offset, molar tooth, and REM, ECG signals can be used to assist the observation of sleep physiological condition, such as, ANS state and heart activity, and EDA can provide information of sleep stage. Besides, if further including the optical sensor, it will be able to acquire oxygen saturation for determining hypopnea, and/or including the motion sensing element, such as, accelerator, it will able to provide information of body movement, and/or including microphone, it will be able to detecting snore. Accordingly, simply through mounting the device on the ear and/or the head, plenty of physiological information during sleep can be obtained almost without burden.

In the aforesaid, through providing a breathing guiding in the neurofeedback section, the wearable physiological monitoring device of the present invention can improve the user's concentration during performing the nurofeedback process according to the present invention, and at the same time, the effect of neurofeedback also can be enhanced due to the complement between these two processes, resulting in magnifying the total effect. And, through the head-worn structure and/or the ear-worn structure, the mounting of the device and the setting of electrodes and/or sensors can be completed at the same time, it not only provides convenient, but also improves mobility. Besides, due to being implemented as wearable type, the device of the present invention is also suitable to be used as brain computer interface which further increases the usage value.

1-15. (canceled)

16. A wearable physiological monitoring device, for providing brain activity information and deciding a breathing guiding signal in a neurofeedback section, for being a basis for a user to perform a self-regulation about brain function, thereby achieving a neurofeedback loop, the device comprising:
plural electroencephalographic (EEG) electrodes;
a heart rate sensing unit;
wearable structure, combined with the plural EEG electrodes and the heart rate sensing unit, wherein when the wearable structure is mounted on the head and/or an ear of the user, the plural EEG electrodes are located on the head and/or the ear where are capable of achieving an EEG signal acquisition loop, and the heart rate sensing unit is located on the head and/or the ear where is capable of acquiring heart beat sequence; and
a physiological signal acquisition circuitry, for acquiring EEG signals via the plural EEG electrodes, and obtaining the heart beat sequence via the heart rate sensing unit,
wherein in the neurofeedback section,
the EEG signals are used to be a basis for generating brain activity related information for providing to the user;
the heart beat sequence is used to perform a respiratory behavior related analysis so as to obtain an analysis result, and the analysis result is then used as a basis to provide and/or adjust the breathing guiding signal; and
the user performs a self-regulation based on the brain activity related information and performs a respiratory behavior based on the breathing guiding signal, so that commonly through the self-regulation about brain and through the respiratory behavior influences autonomous nervous system (ANS), an influence on brain function is achieved.

17. The device as claimed in claim 16, wherein the wearable structure is implemented to be a head-worn structure and/or an ear-worn structure.

18. The device as claimed in claim 16, further comprising a housing combined with the wearable structure for accommodating at least a portion of the physiological signal acquisition circuitry.

19. The device as claimed in claim 18, wherein the housing is implemented to have at least an EEG electrode mounted thereon.

20. The device as claimed in claim 16, wherein the heart rate sensing unit is implemented to be optical sensor for acquiring consecutive pulse variations, so as to obtain the heart beat sequence.

21. The device as claimed in claim 16, wherein the heart rate sensing unit is implemented as a first ECG electrode and a second ECG electrode for acquiring ECG signals, so as to obtain the heart beat sequence, and the first ECG electrode is configured to locate at a position of the wearable structure where contacts the skin of the head and/or the ear, and the second ECG electrode is configured to locate at a position of the device where is capable of being contacted by an upper limb of the user while the device is mounted on the head and/or the ear, or the second ECG electrode is carried by a finger-worn structure for mounting on a finger.

22. (canceled)

23. The device as claimed in claim 21, wherein the first ECG electrode is implemented as one of the plural EEG electrodes.

24-25. (canceled)

26. The device as claimed in claim 16, wherein the wearable structure is implemented to be two ear-worn structures for respectively mounting on two ears, and the plural EEG electrodes are located at the ears or around the ears where are capable of achieving EEG signal acquisition loops of different brain portions, and the brain activity related information is implemented to be a correlation information of brain activities among different brain portions.

27. The device as claimed in claim 16, wherein the brain activity related information together with the analysis result of respiratory behavior are used as a basis of providing and/or adjusting the breathing guiding signal, and the breathing guiding signal and the brain activity related information are provided to the user through a perceptible signal producing source.

28. (canceled)

29. The device as claimed in claim 28, wherein the perceptible signal producing source is configured to provide at least one of visually perceptible signal and auditory perceptible signal, and wherein the perceptible signal producing source is one of an independent lighting object and a device with displaying and/or sounding function, or the perceptible signal producing source is implemented to combine with the physiological monitoring device.

30-31. (canceled)

32. A wearable physiological monitoring device, comprising:
plural EEG electrodes, which are implemented to be dry electrodes;
an optical sensor;
an ear-worn structure, comprising an ear-plug structure or an ear-hook structure, and an ear-clamp structure, wherein the ear-plug structure or the ear-hook structure is configured to mount on an ear of a user, and the ear-clamp structure is configured to clamp an ear portion of the ear;
a physiological signal acquisition circuitry, for acquiring EEG signals via the plural EEG electrodes, and obtaining heart rate via the optical sensor,
wherein
at least one of the plural EEG electrodes and the optical sensor are mounted on the inner side the ear-clamp structure together, and at least another of the plural EEG electrodes is mounted on the ear-plug structure or the ear-hook structure; and
when the ear-clamp structure is configured to clamp on the ear portion, the EEG electrode at the inner side contacts the skin of the ear portion to achieve an EEG signal acquisition loop, and the optical sensor acquires heart beat sequence from the ear portion.

33. The device as claimed in claim 32, wherein the optical sensor includes a light emitting element and a light receiving element for being mounted on the ear-clamp structure so as to acquire heart beat sequence in a transmission manner or in a reflectance manner.

34-35. (canceled)

36. The device as claimed in claim 32, further comprising a first ECG electrode and a second ECG electrode for acquiring ECG signals, wherein the first ECG electrode is configured to locate at a position of the ear-worn structure where contacts the skin of the ear or around the ear, and the second ECG electrode is configured to locate at a position of the device where is capable of being contacted by an upper limb of the user while the device is mounted on the ear.

37. (canceled)

38. The device as claimed in claim 36, wherein the first ECG electrode is implemented to be one of the plural EEG electrodes.

39. (canceled)

40. The device as claimed in claim 32, wherein the device is used as a brain-machine interface.

41. The device as claimed in claim 39, further comprising a movement sensing element for detecting a movement of the user's ear, head, or body.

42-51. (canceled)

52. A wearable physiological monitoring device, comprising:
plural EEG electrodes;
an optical sensor;
an ear-plug structure and an ear-hook structure, wherein the ear-plug structure is configured to mount inside an ear of a user and the ear-hook structure is configured to hook on the ear; and
a physiological signal acquisition circuitry, for acquiring EEG signals via the plural EEG electrodes, and obtaining heart rate via the optical sensor,
wherein
at least one of the plural EEG electrodes is mounted on the ear-plug structure, and at least another of the plural EEG electrodes is mounted on the ear-hook structure;
the optical sensor is mounted on the ear-plug structure; and
when the ear-plug structure and the ear-hook structure are mounted on the ear simultaneously, the plural EEG electrodes contacts the skin of the ear and/or around the ear for achieving an EEG signal acquisition loop, and the optical sensor acquires heart beat sequence from the ear.

53. A wearable physiological detection device, comprising:
plural EEG electrodes;
an optical sensor;
an ear-plug structure, mounted inside an ear of a user; and
a physiological signal acquisition circuitry, for acquiring EEG signals via the plural EEG electrodes, and obtaining heart rate via the optical sensor,
wherein
at least two of the plural EEG electrodes and the optical sensor are mounted on the ear-plug structure; and
when the ear-plug structure is mounted inside the ear, at least one of EEG electrodes which are mounted on the ear-plug structure is configured to contact the canal of the ear, the opening of canal, and/or the inferior concha of the ear for achieving an EEG signal acquisition loop, and the optical sensor acquires heart beat sequence from the ear.
